(19) United States
(12) Patent Application Publication
(10) Pub. No.: US 2007/0118222 A1
(43) Pub. Date: May 24, 2007

(54) Intervertebral devices and methods

(75) Inventor: Philipp Lang, Lexington, MA (US)

Correspondence Address: Philipp Lang, M.D., MBA;Vertegen, Inc., 7 Fair Oaks Terrace, Lexington, MA, 02421, US

(73) Assignee: Vertegen, Inc.

(21) Appl. No.: 11/602,763

(22) Filed: Nov. 21, 2006

Related U.S. Application Data
(60) Provisional application No. 60/740,326, filed on Nov. 21, 2005.

(51) Int. Cl. A61F 2/44

(52) U.S. Cl. 623/17.12

(57) ABSTRACT

An expandable device for spinal fusion or vertebral disc replacement that is inserted via a minimally invasive surgery approach and that is expanded inside the vertebral disc space. The device is configured to expand in at least one of a medial, a lateral, an anterior, a posterior, a superior and an inferior direction and to restrict further expansion of at least one of the medial, lateral, anterior and posterior direction after reaching a maximum allowing further expansion in superior or inferior direction.

### CROSS-REFERENCE TO RELATED APPLICATION

This application claims the benefit of U.S. Provisional Application No. 60/740,326, filed on Nov. 21, 2005. The disclosure of the above application is incorporated herein by reference.

### BACKGROUND OF THE INVENTION

1. Field of the Invention

Embodiments of the invention generally relate to functional spinal device for insertion into an intervertebral disk space between adjacent vertebrae. More specifically, embodiments of the invention relate to an expandable and/or collapsible artificial intervertebral device that can be inserted via a minimally invasive surgical approach.

2. Description of the Related Art

Seven cervical, 12 thoracic, and 5 lumbar vertebrae form the normal human spine. Intervertebral discs reside between adjacent vertebrae with two exceptions. First, the articulation between the first two cervical vertebrae does not contain a disc. Second, a disc lies between the last lumbar vertebra and the sacrum (a portion of the pelvis). The spine supports the body, and protects the spinal cord and nerves. The vertebrae of the spine are also supported by ligaments, tendons, and muscles, which allow movement (flexion, extension, lateral bending, and rotation). Motion between vertebrae occurs through the disc and two facet joints. The disc lies in the front or anterior portion of the spine. The facet joints lie laterally on either side of the posterior portion of the spine. The human intervertebral disc is an oval to kidney bean shaped structure of variable size depending on the location in the spine.

The human spine is a highly flexible structure capable of a high degree of curvature and twist in nearly every direction. However, genetic or developmental irregularities, trauma, chronic stress, and degenerative wear can result in spinal pathologies for which surgical intervention may be necessary. In cases of deterioration, disease, or injury, a spinal disc may be removed from a human spine. A disc may become damaged or diseased, reducing intervertebral separation. Such disruption to the natural intervertebral separation may produce pain, which may be alleviated by removal of the disc and maintenance of the natural separation distance. In cases of chronic back pain resulting from a degenerated or herniated disc, removal of the disc becomes medically necessary. In some cases, a damaged disc may be replaced with a disc prosthesis intended to duplicate the function of a natural spinal disc. In other cases, it may be desirable to fuse adjacent vertebrae of a human spine together after removal of a disc. This procedure is generally referred to as “intervertebral fusion”. Intervertebral fusion has been accomplished with a variety of techniques and instruments, for example structural bone or a fusion cage filled with bone graft material is placed within the space where the spinal disc once resided. Multiple cages or bony grafts may be used within that space. Cages have been generally successful in promoting fusion and approximating proper disc height. Despite such developments in the art, there remains a need for an effective intervertebral device and, in particular, a device which may be introduced into the patient's body through a relatively small incision and that could be expanded from within the intervertebral space to allow restoration of disc space height.

### BRIEF SUMMARY OF THE INVENTION

The present invention relates to an expandable intervertebral device or method for disc replacement or spinal fusion. In one embodiment of the invention, is a device for stabilizing two adjacent vertebrae configured to expand in situ into an intervertebral disc space in at least one of a medial, a lateral, an anterior, a posterior, a superior and an inferior direction and wherein said structure restricts further expansion of at least one of the medial, lateral, anterior and posterior direction after reaching a maximum allowing further expansion in superior or inferior direction and wherein said structure defines a hollow cavity. In one embodiment one or more membranes cover at least part of the hollow cavity. The device may be collapsible. The structure may comprise one or more elements selected form the group consisting of rod-like, bar-like, pillar-like, column-like, sheet-like, pane-like, mesh-like, net-like, lattice-like, ring-like, spiral-like, coil-like, strut-like element. The elements may be assembled in a first orientation and adopt a second orientation in situ allowing expansion or collapse of the structure. The elements may change orientation, may be interconnected, interdigitated, superposed.

In a preferred embodiment, the device is inserted into an intervertebral disk space in a collapsible configuration and is then expanded in situ in an expanded configuration. The expanded configuration may have a final volume sized to consume at least a portion of the intervertebral disk space. The medial, lateral and the anterior, posterior dimensions of the expanded configuration may substantially correspond to at least a portion of a vertebral endplate. The medial, lateral dimensions and the anterior, posterior dimensions of expanded configuration preferably remain unchanged under loading conditions. The anterior and posterior dimensions of the expanded configuration may reestablish a normal curvature of the spine.

In another embodiment, at least one membrane is covering the entirety of the cavity and defines at least one sealed cavity. The membrane may be bioresorbable and/or semi-permeable.

In one embodiment, a filling material is delivered within the hollow cavity of the device or within the membrane. The filling material may include a hardenable material. The filling material may be a compressible liquid or gel or a non-compressible liquid or gel. In another embodiment, the filling material may comprise at least one osteobiologic material selected from the group consisting of BMP/bone morphogenetic proteins, LMP/LIM mineralization protein, and DBM/Demineralized bone matrix, growth differentiation factors (GDF), transforming growth factors (TGF), hydroxyapatite, tri-calcium phosphate (TCP), bioactive glass, calcium phosphate, calcium sulfates, collagen, alginate. The filling material may be solid or semisolid. The filling material may comprise comprises a material selected from the group consisting of hydroxyaptite spheres, plastic spheres, polymeric, ceramic and metal

The structure of the device may be self-expanding. One or more elements may be made of a shape memory material, such as Nitinol and the shape memory material may betemperature responsive. One or more elements may be bioresorbable. One or more elements may be made of a material that selected from a group of corrosive metal or corrosive metal alloys. Once expanded, the cross-sectional shape of the expanded configuration may be kidney-shaped, C-shaped, rectangular, square, cylindrical, capsule, U-shaped, V-shaped, X-shaped, oval-like, spherical and “O” or donut shaped and the footprint may substantially correspond to a perimeter of two adjacent vertebral endplates. In another embodiment, the structure of the device may have regions of differential rigidity or extensibility. The rigidity of an anterior area of the outer structure may be lesser than the rigidity of a posterior area of said outer structure and whereupon filling the cavity of said device expansion of the anterior area may be greater than expansion of the posterior area.

In one embodiment of the invention, the device is introduced into the intervertebral disk space in a collapsible configuration via a hollow catheter or cannula and using a minimally invasive approach. Part of or the entire device structure may be removed after injection of the filling material in a collapsible configuration and via a catheter or cannula.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited. Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims.

In a preferred embodiment, the intervertebral device has an outer expandable structure, which defines a hollow cavity. The outer structure restricts expansion or extension of at least one or more directions. In the spine, this limitation or restriction of extension or expansion occurs typically in antero-posterior and medio-lateral direction, or both. The device may be expandable and collapsible. In another preferred embodiment, an inner membrane covers part of the inner hollow cavity.

In one embodiment, the device can be composed of one or more components. These components can have different shape and function. The components can be composed of different materials, e.g. metal, metal alloys, nitinol, liquid metal, ceramics, carbon based materials, plastics, polymers, polyethylenes, polyurethanes, and the like. Teflon based materials can be used. Biocompatible material such as ePTFE and Dacron.™. may also be used. The materials can be in a solid, semi-solid, gel-like, and fluid-state. The materials can be elastic or rigid. The materials can be compressible and non-compressible. The materials can be expandable.

A wide-variety of metals are useful in the practice of the present invention, and can be selected based on any criteria. For example, material selection can be based on resiliency to impart a desired degree of rigidity. Non-limiting examples of suitable metals include silver, gold, platinum, palladium, iridium, copper, tin, lead, antimony, bismuth, zinc, titanium, cobalt, stainless steel, nickel, iron alloys, cobalt alloys, such as Elgiloy®, a cobalt-chromium-nickel alloy, and MP35N, a nickel-cobalt-chromium-molybdenum alloy, and Nitinol™, a nickel-titanium alloy, aluminum, manganese, iron, tantalum, crystal free metals, such as Liquidmetal® alloys (available from LiquidMetal Technologies, www.liquidmetal.com), other metals that can slowly form polyvalent metal ions, for example to inhibit calcification of implanted substrates in contact with a patient's bodily fluids or tissues, and combinations thereof.

Suitable synthetic polymers include, without limitation, polyamides (e.g., nylon), polyesters, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, polyether ether ketones, ethylene vinyl acetates, polysulfones, nitrocelluloses, similar copolymers and mixtures thereof. Bioresorbable synthetic polymers can also be used such as dextran, hydroxyethyl starch, derivatives of gelatin, polyvinylpyrrolidone, polyvinyl alcohol, poly[N-(2-hydroxypropyl) methacrylamide], poly(hydroxy acids), poly(epsilon-caprolactone), polylactic acid, polyglycolic acid, poly(dimethyl glycolic acid), poly(hydroxy butyrate), and similar copolymers can also be used.

Other materials would also be appropriate, for example, the polyketone known as polyetheretherketone (PEEK™). This includes the material PEEK 450G, which is an unfilled PEEK approved for medical implantation available from Victrex of Lancashire, Great Britain. (Victrex is located at www.matweb.com or see Boedeker www.boedeker.com). Other sources of this material include Gharda located in Panoli, India (www.ghardapolymers.com).

It should be noted that the material selected might also be filled. For example, other grades of PEEK are also available and contemplated, such as 30% glass-filled or 30% carbon filled, provided such materials are cleared for use in implantable devices by the FDA, or other regulatory body. Glass filled PEEK reduces the expansion rate and increases the flexural modulus of PEEK relative to that portion which is unfilled. The resulting product is known to be ideal for improved strength, stiffness, or stability. Carbon filled PEEK is known to enhance the compressive strength and stiffniess of PEEK and lower its expansion rate. Carbon filled PEEK offers wear resistance and load carrying capability.

The device can also be comprised of polyetherketoneketone (PEKK). Other materials that can be used include polyetherketone (PEK), polyetherketoneetherketoneketone (PEKEKK), and polyetheretherketoneketone (PEEKK), and generally a polyaryletheretherketone. Further other polyketones can be used as well as other thermoplastics.

As will be appreciated, other suitable similarly biocompatible thermoplastic or thermoplastic polycondensate materials that resist fatigue, have good memory, are flexible, and/or deflectable have very low moisture absorption, and good wear and/or abrasion resistance, can be used without departing from the scope of the invention.

Reference to appropriate polymers that can be used for the device made to the following documents, all of which are incorporated herein by reference. These documents include: PCT Publication WO 02/02158 A1, dated Jan. 10, 2002 and entitled Bio-Compatible Polymeric Materials; PCT Publication WO 02/00275 A1, dated Jan. 3, 2002 and entitled Bio-Compatible Polymeric Materials; and PCT Publication WO 02/00270 A1, dated Jan. 3, 2002 and entitled Bio-Compatible Polymeric Materials.

The substrate can be textured or made porous by either physical abrasion or chemical alteration to facilitate incorporation of the metal coating. Other processes are also appropriate, such as extrusion, injection, compression molding and/or machining techniques. Typically, the polymer is chosen for its physical and mechanical properties and is suitable for carrying and spreading the physical load between the vertebral surfaces.

Polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and sulfonated polymers can be used alone or, more typically, in combination.

If a hydrogel is used, polymers can be at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Acidic groups can be carboxylic acid groups, sulfonic acid groups, and halogenated (preferably fluorinated) alcohol groups. Some examples of polymers with basic side groups that can react with anions can include poly(vinyl amines), poly(vinyl pyridine), and poly(vinyl imidazole).

Water soluble polymers with charged side groups are cross-linked by reacting the polymer with an aqueous solution containing multivalent ions of the opposite charge, either multivalent cations if the polymer has acidic side groups, or multivalent anions if the polymer has basic side groups. Cations for cross-linking the polymers with acidic side groups to form a hydrogel include divalent and trivalent cations such as copper, calcium, aluminum, magnesium, and strontium. Aqueous solutions of the salts of these cations are added to the polymers to form soft, highly swollen hydrogels.

Anions for cross-linking the polymers to form a hydrogel include divalent and trivalent anions such as low molecular weight dicarboxylate ions, terepthalate ions, sulfate ions, and carbonate ions. Aqueous solutions of the salts of these anions are added to the polymers to form soft, highly swollen hydrogels, as described with respect to cations. The hydrogel pore size can be designed to limit the passing of antibodies into the hydrogel, while allowing the supply of nutrients.

Alginates or chitosan, which fall into the category of ionic polysaccharides, may be employed and can be utilized to suspend living cells. U.S. Pat. No. 4,352,883 describes the ionic cross-linking of alginate with divalent cations, in water, at room temperature, to form a hydrogel matrix.

Optionally, blood, marrow, stem or other cells can be mixed with an alginate solution. The solution can then be delivered into a hollow cavity formed by the device. The solution can optionally solidify in the presence of calcim ions. The solution can also be delivered to the device prior to implantation and solidified in an external solution containing calcium ions.

The hydrogel can include or be compose of alginate. Alginate can be gelled under mild conditions, allowing cell immobilization with little damage. Binding of Mg.sup.2+ and monovalent ions to alginate does not induce gelation of alginate in aqueous solution. However, exposure of alginate to soluble calcium leads to a preferential binding of calcium and subsequent gelling. These gentle gelling conditions are in contrast to the large temperature or solvent changes typically required to induce similar phase changes in most materials.

More than one metal and/or polymer can be used in combination with each other. For example, one or more metal-containing substrates can be coated with polymers in one or more regions or, alternatively, one or more polymer-containing substrate can be coated in one or more regions with one or more metals.

The expandable structure can be composed of one or more elements. In a preferred embodiment, multiple elements can be used. The elements can be arranged in a substantially parallel manner or can be arranged in a substantially non-parallel manner. The elements can be located in one or more layers. The individual elements can be interconnected within the same or adjacent layers. Layers can, optionally, cross-over or be woven. The elements of the expandable structure can be rod-like, bar-like, pillar-like, column-like, sheet-like, pane-like, mesh-like, net-like, lattice-like, ring-like, spiral-like, coil-like, and strut-like. One or more types of elements can be used. Elements can be arranged in one or more layers. If more than one layer of elements is utilized, the layers can be inserted into the body simultaneously. Alternatively, a second and subsequent layers can be delivered sequentially, after the first layer has been placed inside the body, e.g. an intervertebral disc space. A multi-layer configuration can add biomechanical strength, and can add additional strength for limiting or restring expansion or extension of the device in one or more directions, e.g. antero-posterior and/or mediolateral. A multi-layer configuration can also provide an improved sealing effect if the expandable or collapsible structure includes a hollow cavity to allow for introduction of filling materials.

If different structural elements are used within the expandable and collapsible device, these can, for example, include rings and vertical bars. In one example, rings can be inserted initially, and can be optionally connected via a membrane (see below). A second layer including rods can be introduced at the same time or subsequently. In the collapsed state, the rings can be located very close or on top of each other. In the expanded state, the distance between the rings increases. In the collapsed state, the rods can be oriented substantially horizontally. In the expanded state the rods can be oriented substantially vertically.

The elements of the expandable structure can have a constant or variable thickness. In one embodiment, the central portion of these elements can be thicker than the proximal and distal portion. In another embodiment, the elements can be thicker in antero-posterior dimension and less thick in medio-lateral dimension. This arrangement can be favorable to allow for lateral bending, while restricting flexion and extension. In another embodiment of the invention, the elements can be thicker in medio-lateral dimension than in antero-posterior dimension, thereby allowing for more lateral bending and less or no flexion and extension.

In another embodiment of the invention, more elements or thicker elements or both can be present in parts of the expandable structure. For example, more elements or thicker elements or both can be present in the posterior region of the expandable structure thereby limiting or restricting extension. In another embodiment, more elements or thicker elements or both can be present in the anterior region of the expandable structure thereby limiting or restricting flexion.

In another embodiment, more elements or thicker elements or both can be present in the left and right region of the expandable structure thereby limiting or restricting lateral bending. Thus, the arrangement and composite stiffness of the elements can be optimized for a particular vertebral level and patient. Moreover, the arrangement of the elements can be optimized to enable or restrict rotation at a motion segment.

The elements can have constant radii or variable radii. The cross-section of the elements can be substantially round, substantially elliptical, substantially triangular, substantially rectangular, substantially polygonal, substantially irregular. The elements can have the same length or variable length. For example, if rods are used, these can be longer anteriorly than posteriorly, thereby re-establishing a normal lumbar lordosis with a greater disc height anteriorly when compared to posteriorly. One or more ends of each element can be flat, rounded, or sharp. More than one spike can be present on one or more ends of one or more elements. The ends can also have an irregular shape with several sharp edges. A sharp or spike like configuration can be of use to assist with tissue anchoring, e.g. against a vertebral endplate.

One or more elements can have a memory shape. A memory shape can assist to help expand the expandable structure once inserted into the body, e.g. an intervertebral disc space. The shape-memory/shape restoring properties of alloys such as Nitinol make them preferred.

In a preferred embodiment, the elements are assembled in a first orientation and adopt a second orientation in situ allowing expansion or collapse of the structure. Thus, the elements can change orientation. They can also be interconnected, interdigitated or superimposed.

The overall outer configuration of the expandable structure can be substantially round, substantially elliptical, substantially triangular, substantially rectangular, substantially polygonal, substantially irregular, substantially horseshoe-shaped, substantially kidney-shaped, or combinations thereof in one or more dimensions.

In a preferred embodiment, the expandable structure can have an external shape that is substantially elliptical and that follows substantially the outline of the upper and lower endplate.

In a preferred embodiment, the expandable or collapsible structure is delivered in a collapsed state into the body, for example an intervertebral disc space. Once inserted it can be expanded in situ. Alternatively, the expandable or collapsible structure can be expanded outside the body and inserted into the body in an expanded state. Insertion in a collapsed state is typically preferred since it allows for smaller incision size and smaller access and, with that, less invasive surgery. In one embodiment, the device is delivered in minimally invasive technique, with an incision size of less than 8 cm, more preferred less than 5 cm, more preferred less than 3 cm and, even more preferred, less than 1 cm. Once in situ, the expandable or collapsible structure can be compressible and/or allow for elastic deformation or non-compressible and/or not allow for elastic deformation. A compressible and/or elastically deformable structure would be preferred if a disc replacement like device is desired. A non-compressible and/or non-elastically deformable structure would typically be preferred of a spinal fusion device is desired.

As the spine, and with it the device is loaded, forces that are transmitted in superior to inferior direction will result in forces in lateral, anterior and posterior direction. The device is designed to be strong enough to withstand these forces and, with that, maintain the device height and the resultant height of the intervertebral disc space. If a fusion device is used, the height of the device will not change and will be maintained at constant values. If a disc, nuclear or annular replacement device is used, the device height and shape will preferably deform minimally, similar to the deformation seen in a normal, healthy intervertebral disc.

In one embodiment the device is composed of a degradable material (e.g., resorbable, bioresorbable, degradable, absorbable, bioabsorbable, erodible, or bioerodible). Thus, over time, the expandable or collapsible structure can be resorbed. In particular implementations, the device is composed of a metal or metal alloys designed to allow corrosion at a preferably predetermined rate. Corrosion can be used for absorption and disappearance of the device over time without endangering support of the disc space.

The expandable and/or collapsible structure can optionally form a hollow cavity. The hollow cavity can be used to place a second device of similar or different design. The second device can, optionally, also be expandable or collapsible.

Another material can be introduced into the hollow cavity formed by the expandable or collapsible structure. By introducing the other material, the expandable or collapsible structure can be progressively expanded. Since expansion is, for example, limited or restricted in antero-posterior and medio-lateral dimension, the expansion will typically occur in superior or inferior direction or both.

In a preferred embodiment, the expandable or collapsible structure is designed so that it can withstand pressures from within the hollow cavity in one or more directions, e.g. antero-posteriorly and/or medio-laterally, thereby restricting or limiting expansion in these directions while allowing expansion in superior or inferior direction or both.

By controlling the amount of material introduced into the hollow cavity, the degree of expansion can be controlled. In this manner, the height or other dimension of the expandable structure can be optimized to approach that, for example, of a normal intervertebral disk for a given vertebral level.

Materials that can be introduced into the hollow cavity include, but are not limited to fluid-like materials, semi-fluid-like materials, gel-like materials, including hydrogels, mesh like material, sphere-like materials (including solid sphere-like materials (e.g. made of plastics, metal, or metal alloy), fluid filled sphere-like materials, elastic sphere-like materials, non-elastic sphere-like materials) tissue matrix-like materials, chitosan-like materials, bone allograft (e.g. in pellet, ground, or soluble form), bone autograft (e.g. in pellet, ground, or soluble form), blood, blood clot, serum, cells, proteins, other osteobiologics, drugs, solid materials in various shapes and sizes (including metal, metal alloys memory shape materials (e.g. Nitinol), liquid metal, ceramics, carbon based materials, plastics, polymers, polyethylenes, polyurethanes, teflon based materials bioresorbable materials). These materials can be used alone or in combination. The material can be bioresorbable or, if a metal or metal alloy is used, the material can be corrosive. Thus, over time, the material can be replaced with other biological material formed by the body such as collagen or fibrous tissue. Solid materials can, for example, include mesh like materials or materials in expandable or collapsible shape, e.g. spring or coil-shaped like materials. Solid materials can be hollow on the inside. These materials can optionally be drug coated, drug carrying, or drug encapsulated.

The materials can fill portions of the hollow cavity formed by the expandable or collapsible device. In a preferred embodiment, the materials will fill the entire hollow cavity formed by the expandable or collapsible device.

The materials can be introduced into the hollow cavity in situ or external to the patient. In situ introduction into the hollow cavity, typically in conjunction with in situ expansion of the expandable and collapsible structure is typically preferred since it can allow for smaller incision size and smaller access.

Optionally, the expandable and collapsible structure can be introduced into the intervertebral disc space via a hollow surgical port or access system. Optionally, the materials can be introduced into the hollow cavity formed by the expandable or collapsible structure via, for example, a hollow cannula or needle-like system.

If the material itself is hollow, it can, optionally, be introduced into the hollow cavity via a rod-like introducer with a diameter smaller than the inner diameter of the material.

The material introduced into the hollow cavity can be compressible and/or elastic or non-compressible and/or non-elastic. A compressible and/or elastic material would be preferred if a disc replacement like device is desired. A non-compressible and/or non-elastic material would typically be preferred of a spinal fusion device is desired.

In one implementation of the invention, the hollow cavity is compartmentalized, for example with use of subsegments created by the arrangement of the elements of the expandable or collapsible structure. Alternatively, compartmentalization can be achieved with use of one or more membranes or membrane like structures. Compartmentalization of the hollow structure can allow using different materials with different material properties in one or more compartments, thereby, for example, influencing the biomechanical behavior of the composite device. In this manner, for example, flexion and extension can be facilitated while, for example, limiting or restricting lateral bending or rotation. Someone skilled in the art will recognize many possible modifications of this concept.

In one embodiment, at least one membrane or membrane like structures can be used in conjunction with the expandable or collapsible structure. These membranes can, for example, limit or restrict of material introduced into the hollow cavity outside the hollow cavity, in particular during expansion or loading of the expandable or collapsible structure.

A single, dual or multiple membrane design can be used. A first membrane can be located peripheral to a second membrane. The membranes can be used to create one or more compartments inside the hollow cavity. For example, two or more adjoining membranes can create a superior and an inferior compartment. Alternatively, two or more adjoining membranes can create an anterior and a posterior compartment. One, two, three, four or more compartments can be produced with use of one or more membranes.

In one implementation of the invention, the membrane covers the entire hollow cavity and is sealed. The membrane expands upon introduction, typically by injection of a filler material or load bearing material, such that upon expansion, the membrane will generally adapt and conform three-dimensionally to the dimensions of the hollow cavity defined within the outer support structure. The membranes can, optionally, be self-sealing, for example via their material properties. This is a preferred embodiment if fluids or gels and the like are introduced into the hollow cavity formed by the expandable or collapsible structure. Self-sealing can, for example, be achieved by absorption of tissue water and swelling and expansion of the membrane material. Alternatively, the membranes can be sealed in situ, for example, with use of injection of a sealing agent. The sealing agent can be biologic (e.g. fibrin-like glue) or non-biologic. Someone skilled in the art will recognize a large number of sealing agents, also dependent on the type of membrane used.

One or more membranes can cover the entire hollow cavity. One or more membranes can cover portions of the hollow cavity. If more than one membrane is used, these can be arranged symmetrically or asymmetrically. If more than one membrane is used, these can have the same dimensions in antero-posterior and/or medio-lateral and/or supero-inferior direction or they can have different dimensions in one or more directions.

The membrane(s) may be porous to permit osteoincorporation and/or bony ingrowth. The membrane(s) may consist of a biocompatible and bio-inert polymer material, such as polyurethane, silicone, or polycarbonate-polyurethane (e.g., Corethane). Non-limiting examples of specially formulated biodegradable polyurethanes are disclosed in the following exemplary published materials, the contents of which is fully incorporated herein by reference: Gorna, K., and Gogolewski, S., “In vitro degradation of novel medical biodegradable aliphatic polyurethanes based on e-caprolactone and Pluronics RTM with various hydrophilicities,” Polymer Degradation and Stability 75 (2002), pp. 113-122; and Goma, K., and Gogolewski, S., Biodegradable porous polyurethane scaffolds for tissue repair and regeneration, J Biomed Mater Res A. 2006 October; 79(1):128-38. In another embodiment, the membrane can exert a filtration effect, by limiting passage, for example, only to molecules of a particular size or charge.

Membrane may be bio-resorbable. Membranes comprising bio-resorbable polymers may be transformed by physiological conditions into substances that are non-harmful and biologically compatible or naturally occurring in the body. These substances may remain in the patient or be expelled from the body via metabolic activity. Membranes may also be a porous or selectively porous allowing fluid to move in and out of the cavity.

Resorbable portions of the containment device may be formed from polymer films made from synthetic materials, naturally occurring materials, modified naturally occurring materials and combinations thereof. For instance, materials suitable for synthesizing polymer films for the containment device may be formed wholly or in part from biodegradable polyurethane based on epsilon.-caprolactone (e.g., polycaprolactone-based elastomers), which can be transformed into a film by solution casting (e.g., dip coating). Another suitable polyurethane is based on polycaprolactone-polyethylene oxide-polypropylene oxide-polyethylene oxide (Pluronic). The Pluronic may be dissolved, for example, in tetrahydrofuran.

Resorbable membranes may also include polymers such as highly purified polyhydroxyacids, polyamines, polyaminoacids, copolymers of amino acids and glutamic acid, polyorthoesters, polyanhydrides, polyamides, polydioxanone, polydioxanediones, polyesteramides, polymalic acid, polyesters of diols and oxalic and/or succinic acids, polycaprolactone, copolyoxalates, polycarbonates or poly(glutamic-co-leucine). Preferably used polyhydroxyacids may comprise polycaprolactone, poly(L-lactide), poly(D-lactide), poly(L/D-lactide), poly(L/DL-lactide) polyglycolide, copolymers of lactide and glycolide of various compositions, copolymers of said lactides and/or glycolide with other polyesters, copolymers of glycolide and trimethylene carbonate, poly(glycolide-co-trimethylene carbonate), polyhydroxybutyrate, polyhydroxyvalerate, copolymers of hydroxybutyrate and hydroxyvalerate of various compositions. Other materials which may be used as additives are composite systems containing resorbable polymeric matrix and resorbable glasses and ceramics based e.g. on tricalcium phosphate and/or hydroxyapatite, admixed to the polymer before processing.

Polymer films may be formulated for different degradation rates in vivo. A polymer film may be designed to substantially degrade in a matter months, weeks, or days.

The expandable or collapsible structure or the material introduced into the hollow cavity formed by the expandable or collapsible structure or both can be formed in whole or, more typically, in parts by mesh-like elements or materials. Such mesh-like elements or materials can include mesh or mesh-like with constant material thickness, mesh or mesh-like with variable material thickness, mesh or mesh-like composed of non-elastic material, mesh or mesh-like composed of elastic material, mesh or mesh-like with non-elastic properties, mesh or mesh-like allowing elastic deformation, e.g. via mesh element orientation or use of elastic material forming mesh or combinations thereof

One or more mesh or mesh-like elements or materials can be employed within the expandable or collapsible structure and/or for filling the hollow cavity. These mesh or mesh-like elements or materials can have the same material thickness, or different material thickness, the same material properties or different material properties, the same biomechanical properties or different biomechanical properties, the same mesh elements orientation or different mesh element orientation, the same anatomic orientation, or different anatomic orientation.

In a most preferred embodiment, the device is expanded once inside the intervertebral disk space. Preferably, the device includes a membrane to receive the injection of a filling material. Such filling material may be initially in the form of a solid, semi-solid or fluid. The solids may take the form of a single structure, e.g., a cord or spherical or cylindrical shaped structure, a plurality of beads or particles, spheres or microspheres of hydroxyapatite, plastics or metal, or a powder so as to be easily deliverable through or within the implanted device. Spheres or microspheres may be filled with fluid, solid, elastic and non-elastic material. The fluids may be in the form of a gel, liquid or other flowable material. In one embodiment, the device is filled with a thick paste of hardening material that can help withstand axial loading. For example, bony putty, BMP preparation, or injectable hydroxyapatite preparation can be used.

Other filling materials may be used with the present invention. Examples of suitable materials include but are not limited to biocompatible materials such as polyurethane, polyurethane foams, hydrophilic polymers, hydrogels, homopolymer hydrogels, copolymer hydrogels, multi-polymer hydrogels, or interpenetrating hydrogels. The materials may also include natural or biologic or bioingeneered material which are autologous, allograft, zenograft. Examples of such biologic materials include but are not limited morselized or block bone, hydroxyapatite, collagen or cross-linked collagen, muscle tissue, fat, cellulose, keratin, cartilage, protein polymers, etc. which may be transplanted or bioengineered materials.

In one embodiment, the device is filled with an osteobiologic material that promotes bone growth. Osteobiologic material includes osteoinductive and/or osteoconductive materials that that can induce and/or support existing or new bone growth. Examples of osteoinductive materials are bone morphogenetics protein (BMP), growth differentiation factors (GDF) and transforming growth factors (TGF) and other growth factors. Growth factors are a wide group of molecules known to starts or enhances a cellular response resulting in a bone formation process. According to the current knowledge, bone morphogenetic proteins (BMP) are the only growth factors known to induce bone formation heterotopically by inducing undifferentiated mesenchymal cells to differentiate into osteoblasts. Examples of osteoconductive material used as a matrix for bone tissue ingrowth are hydroxyapatite, tri-calcium phosphate (TCP), bioactive glass, calcium phosphate, calcium sulfates, collagen, alginate, or combinations thereof. Osteoconductive materials may be resorbable with ingrowth of new-formed bone in the spine of a patient. Examples of osteoconductive and osteoinductive materials are type I collagene which has a three dimensional structure and binds circulating growth factors, demineralized bone matrix (DBM) composed of 90% type I collagene and 10% non-collageneous proteins.

In any of these forms, the material is selectively delivered in an amount to increase the disc volume, pressure and/or height. By expanding the device to its maximum dimension with the filling material, ML and AP expansion is maximized and loading of the device in superoinferior and other directions is possible.

In some embodiments, the device may be impregnated, coated or otherwise delivered with one or more therapeutic agents. The therapeutic agent can facilitate pain reduction, stimulate healing, inhibit scarring, prevent infection, stimulate growth or ingrowth etc . . . , and can include genetically active growth or healing factors. Therapeutic agents may be dispersed in a regulated or time-released fashion.

The device can, optionally, have a sealed design. For example, a membrane can cover the internal perimeter of the expandable or collapsible structure. The membrane can have a sealing effect that prevents or limits, for example, the exit of any fluids, gels, gel-like, mesh-like, sphere-like, mesh-like, osteobiologic or other materials that have been introduced into the optional hollow cavity. The membrane can, optionally, be attached to the expandable or collapsible structure. The membrane can, optionally, also be attached or sealed against the vertebral endplates.

Alternatively, the internal perimeter of the expandable or collapsible structure can be covered by a plastic that can have a sealing effect. The plastic can be inserted together with the expandable or collapsible device or separate from the expandable or collapsible device.

In another embodiment, the elements of the expandable or collapsible structure can have a distance to the next adjacent element that is smaller than the smallest diameter of a material inserted into the hollow cavity. For example, if the expandable or collapsible structure is composed of rod-like elements, the distance between the rod-like elements in the expanded state can be smaller than the smallest diameter of the material inserted into the hollow cavity, e.g. elastically deformable spheres. In this manner, the overall integrity of the device can be maintained, even during various loaded and unloaded states.

In a preferred embodiment, the superior and inferior vertebral endplates can create a natural barrier or seal for the material inserted into the hollow cavity.

The device can include various types of tissue anchors. Tissue anchors can be integrated into the ends of one or more elements forming the expandable or collapsible structure. Tissue anchors can also be separate outward protruding extenders that protrude, for example, from the periphery or from within the expandable or collapsible structure.

The material inserted into the optional hollow cavity can act as a tissue anchor, for example, by creating at least one of a chemical, a mechanical or a structural bond between the material and, typically, one or more vertebral endplates.

Tissue anchors can have various shapes, for example spike-shaped, hook-shaped, ring-shaped, semilunar-shaped, peg-shaped, keel-shaped, U-shaped, ratchet-shaped, etc . . .

The tissue anchors can, optionally, be not outward projecting in the collapsed state of the device and can be outward projecting in the expanded state. For example, as more and more material is inserted into the optional hollow cavity and the internal pressure increases, the expandable structure can change its overall shape thereby exposing the tissue anchors, for example, towards the endplate, with resultant progressive anchoring against the endplates.

The footprint of the device can be similar in part or in whole to the footprint of the vertebral endplate. The footprint of the device can be larger than the vertebral endplate(s) in one or more regions or along its entire perimeter. The footprint of the device can be smaller than the vertebral endplate(s) in one or more regions or along its entire perimeter. A smaller footprint can allow for less invasive placement of the device. A larger footprint can assist with device stabilization between the two endplates.

The expandable or collapsible structures or some extenders extending from it can optionally extend beyond the borders of the vertebral endplate and surround or attach to the anterior, posterior, left and/or right wall of the vertebral body. Such extension to the anterior, posterior, left and/or right wall can help in fixing the device against the vertebral body.

Optionally, the device can be attached to the vertebral body using additional attachment means such as screws, pins and any other attachment means known in the art.

The device can be smaller than a nucleus pulposus, can have the same size as a nucleus pulposus or can be larger than a nucleus pulposus, in whole or in part. The device can be smaller than an annulus fibrosus, can have the same size as an annulus fibrosus or can be larger than an annulus fibrosus, in whole or in part. The device can be smaller than the footprint of a vertebral endplate, can have the same size as the footprint of a vertebral endplate or can be larger than the footprint of a vertebral endplate, in whole or in part. The device can occupy a volume smaller than the intervertebral disc space, same as or similar to a vertebral disc space, or larger than a vertebral disc space, in whole or in part.

In one embodiment, a single expandable or collapsible structure is used or two or more expandable or collapsible structures are used. If two or more expandable or collapsible structures are used, they can be, optionally, interconnected or interdigitated. Two or more expandable or collapsible structures that are each individually smaller than a nucleus pulposus can be beneficial for replacement or repair of the nucleus pulposus. The overall shape of the combined expandable structures can be, for example, coffee bean shaped.

The device dimensions can be optionally adjusted in the expanded state. For example, more material can be introduced into the hollow cavity once the device has been placed and expanded in the intervertebral disc space. One or, if present, more compartments inside the hollow cavity can be filled in this manner. Filling can, optionally, be performed at different filling pressures or with different amounts of filling materials. Thus, by controlling the filling pressure or the amount of filling material within the same compartment or, when present, within multiple compartments, the resultant dimensions, e.g. height, of the device can be controlled. When multiple compartments are present, e.g. one at the anterior aspect of the device and another at the posterior aspect of the device, the anterior and posterior device height can be optimized.

The device dimensions can be adjusted intra-operatively. In one embodiment, the device dimensions can also be adjusted postoperatively. For example, a small access can be made to the device, or, more typically, the hollow cavity within the device, or one or more compartments within the device, and more material can be introduced into the hollow cavity or the compartments. Access can, for example, be gained via a small cannula, scope or other instrument. As previously mentioned, the device can be sealed, self-sealing or a seal can be placed or used after the procedure.

In this manner, the device dimensions and also pressures and forces exerted, for example, onto the endplates, can be optimized after the surgery, with the patient providing feedback. Device dimensions can optionally be adjusted on more than one occasion.

A multi-step adjustment of device height, filling and filling pressures of the hollow cavity or one or more compartments within the device can be beneficial to achieve improved patient tolerance and, ultimately, improved results in pain and function. An abrupt increase in disc height and pressure will typically result in significant pain, since there is no time for adaptation of other spinal and neural elements. With progressive, step-wise adjustment in disc heights, with time for adaptation between each procedure or adjustment, however, restoration of disc height can be better tolerated and more successful.

Device height can be evaluated during different physical activities. Imaging can be used for evaluating device height.

Optionally, pressure measurements can be performed during or after placement of the device, e.g. in the intervertebral disc space. The pressure measurements can, for example be performed at the interface between the device and the vertebral endplate (e.g. to evaluate if the pressure is high enough to achieve a sufficient seal between the endplate and the device) or the pressure measurements can be performed inside the hollow cavity or inside one or more compartments.

An optimal result can be achieved between device expansion, e.g. in superior or inferior direction to resurrect disc height, and intra-device or intra-discal pressure.

Pressure measurements can be performed at the time of the surgery or at a later time in order to evaluate device function and, for example, to optimize filling of the hollow cavity or one or more compartments post-operatively. Pressure measurements can be performed mechanically or electronically or with any other device or method known in the art, currently available or developed in the future.

One or more pressure sensors can be integrated with the device. Pressure readings can be obtained, for example, by connecting a tubing or an electrode to the pressure sensor. In one embodiment, a small chip, an energy storage unit and a transmitting unit, for example using radio frequency or infrared transmission, can be integrated into the device. Pressure readings can be stored and later transmitted or can be transmitted in real time. In this manner, it can be possible to monitor pressure at the device—vertebral body interface or within the device during different physical activities and during resting. This information can be used to further optimize the filling and height of the device. Pressure readings can also be compared to patients' symptoms for further optimization of clinical results.

Various imaging modalities can be used for pre- and postoperative evaluation. These include, but are not limited to: Radiography in one or more planes, discography, CT, MRI, CT or MRI with intrathecal contrast, CT or MRI with intravenous contrast, CT or MRI with intradiscal contrast, ultrasound, nuclear scintigraphy, SPECT and PET.

Pre-operative imaging can be used to select the device that will best fit a particular patient and that will afford the optimal degree of expansion, e.g. in superior and/or inferior direction. Discography or CT or MRI with intradiscal contrast can be used to determine the optimal size of a nuclear replacement or repair device. Pre-operative imaging can also be used to estimate the optimal device height once fully expanded, for example by evaluating disc height of adjacent levels. Pre-operative imaging can also be performed during different types of activities and postures, e.g. lying, standing, flexion, extension, and lateral bending. Pre-operative imaging can also be used to determine the optimal dimensions and performance characteristics of a device and then to (a) either select the device that appears best suited for a patient or (b) manufacture a patient specific device, e.g. using object coordinates and shape information provided by the pre-operative imaging test. Pre-operative imaging can include measurement of the preferred size and dimensions in at least two dimensions or, more preferred, three dimensions. A 3D representation of the shape of the disc and the desired device shape can be helpful to select the best fitting device or to custom manufacture a patient specific device.

Finite element modeling of different loading conditions can optionally be performed to estimate or determine the optimal filling amount and/or pressure of the hollow cavity or compartments within the device.

The device can be designed to achieve vertebral fusion between two or more adjacent vertebral bodies. In this embodiment, the device functions similar to a standard device used for anterior spinal fusion, e.g. a so-called cage device. The device will not be significantly compressible once fully expanded and deployed.

The device height is preferably adapted to achieve a normal or near normal disc height, unless this would result in neurologic impairment. The anterior height will typically differ from the posterior device height as well as the height of the device on the left and right side.

In one embodiment, the device can be selected or designed so that it covers most of the lower and upper endplate once fully expanded.

The device can also be selected or designed as a means of disk augmentation and repair, or disc replacement or augmentation, augmentation, repair or replacement of the nucleus pulposus or augmentation, repair or replacement of the annulus fibrosus.

In these embodiments, the device can be at least partially compressible or elastic. The compressibility or elasticity is typically selected to match that of a normal disc of similar dimensions in a normal vertebral level. The compressibility and elasticity can be selected to allow for normal flexion, extension, rotation and lateral bending.

Optionally, attachment means can secure the device to the endplates or the anterior or posterior or sidewalls of the vertebral body or combinations thereof. In a preferred embodiment, the attachments means get anchored or attached even more tightly into the vertebral body the more the device is being compressed. This can be achieved, for example, by advancing the attachment means or anchors even further towards the vertebral body or endplate as the loading and compression of the device increases.

In the case of a nuclear augmentation, repair or replacement, the device will typically occupy an area similar in size and dimension when compared to the nucleus pulposus. The preferred size can vary depending on the patients' age and the degree of degeneration of the nucleus pulposus. The preferred size can be ascertained with use of a discogram or intradiscal injection of a contrast agent followed by CT or MRI scanning.

In the case of an annular augmentation, repair or replacement, portions or the entire device can optionally be placed over parts of the nucleus pulposus and, optionally, also annular material. The device can then be selected or designed to help prevent future extrusion of annular material, for example via selection of a device with tightly spaced elements, e.g. rods, that make extrusion of annular material through the device difficult.

In one embodiment, a normal or near normal lumbar or cervical lordosis is replicated or restored. As used herein, the term “normal or near normal lordosis” refers to a natural angle between two adjacent vertebral plates within the lumbar or cervical spine segments wherein the distance between the anterior portions of the two adjacent vertebral plates is not smaller than the distance between the posterior portions of the two adjacent vertebral plates.

In another embodiment, a normal or near normal kyphosis is replicated or restored. As used herein, the term “normal or near normal kyphosis” refers to a natural angle between two adjacent vertebral plates within the thoracic spine segment wherein the distance between the anterior portions of the two adjacent vertebral plates is not greater than the distance between the posterior portions of the two adjacent vertebral plates.

The device can be made or can be adapted to help re-establish a normal or near normal lumbar and cervical lordosis or thoracic kyphosis. For example, the height of the device anteriorly can be different from the height of the device posteriorly. Difference in height can, for example, be achieved by different degrees of filling of one or more compartments within the hollow cavity or by different length or shape or thickness of elements of the expandable structure used anteriorly as compared to posteriorly.

Restoring normal or near normal lordosis or kyphosis as well as restoring normal or near normal disc height at the operated level can help improve or restore normal function at adjacent levels. For example, by at least partially restoring normal or near normal disc height, the function of the facet joints with the adjacent levels will be improved thereby improving motion and, possibly, facet related pain.

The device can optionally also be designed to restore normal or near normal function of the nucleus pulposus, annulus fibrosus or the entire disk, in particular when it is designed to be elastic and/or at least partially compressible. As outlined above, the device can optionally be designed to achieve a biomechanical behavior that is substantially similar to that of the disc or portions of the disc.

The device is typically inserted into the body, e.g. an intervertebral disc interspace, in a collapsed state. The device is then expanded. Expansion can occur, for example, via introduction of a filling material into a hollow cavity inside the device. Optionally, a balloon like device or a tissue spreader or a ratchet like device or other mechanical means of distracting the disc space can be used to assist with expansion of the device. Optionally, pedicle screws can be placed and distraction can be achieved via the pedicle screws followed by or concomitant with device insertion and/or expansion.

The device is preferably inserted via a minimally invasive approach, for example using a surgical port. Preferably, the incision size is less than 8 cm, more preferably less than 5 cm, more preferably less than 3 cm and, even more preferably, less than 1 cm.

The device can leave a cavity between outer containment structures to allow for placement of a filling material.

If a balloon has been used for device expansion, the balloon can be progressively deflated as more filling material is introduced. The balloon can optionally be bioresorbable and left in situ. Alternatively, the balloon can be retracted once fully deflated.

A cathether or cannula or other delivery means can be inserted into the device in order to allow for filling of the hollow cavity or the compartments within the device, for example with bone cement, bone putty, polymers, PMMA spheres, and other materials listed above or known in the art. Optionally, the endplates can be drilled or roughened up, for example to induce some bleeding and cell ingrowth.

Intraoperative sizing tools can be used to estimate the preferred size of the device.

I claim:

1. An intervertebral device for stabilizing two adjacent vertebrae in a spine comprising an expandable structure configured to expand in situ into an intervertebral disc space in at least one of a medial, a lateral, an anterior, a posterior, a superior and an inferior direction and wherein said structure restricts further expansion of at least one of the medial, lateral, anterior and posterior direction after reaching a maximum allowing further expansion in superior or inferior direction and wherein said structure defines a hollow cavity.

2. The device according to claim 1 wherein at least one membrane covers at least part of the hollow cavity.

3. The device according to claim 1 wherein the device is collapsible.

4. The device according to claim 1 wherein said structure comprises at least one element selected form the group consisting of rod-like, bar-like, pillar-like, column-like, sheet-like, pane-like, mesh-like, net-like, lattice-like, ring-like, spiral-like, coil-like, strut-like element.

5. The device according to claim 4 wherein said elements are assembled in a first orientation and adopt a second orientation in situ allowing expansion or collapse of the structure.

6. The device according to claim 4 wherein said elements change orientation.

7. The device according to claim 4 wherein elements are interconnected.

8. The device according to claim 4 wherein the elements are interdigitated.

9. The device according to claim 4 wherein the elements are superposed.

10. The device of claim 3 wherein said device is inserted into an intervertebral disk space in a collapsible configuration and wherein the device is expanded in situ in an expanded configuration.

11. The device of claim 10 wherein the expanded configuration has a final volume sized to consume at least a portion of the intervertebral disk space.

12. The device of claim 10 wherein the medial, lateral dimensions and the anterior, posterior dimensions of expanded configuration remain unchanged under loading conditions.

13. The device of claim 2 wherein the at least one membrane is covering the entirety of the cavity and defines at least one sealed cavity.

14. The device of claim 2 wherein the at least one membrane is bioresorbable.

15. The device of claim 1 wherein a filling material is delivered within the hollow cavity of the device.

16. The device of claim 2 wherein a filling material is delivered within the at least one membrane.

17. The device of claim 2 wherein at least one membrane is semi-permeable.

18. The device of claim 10 wherein the medial, lateral and the anterior, posterior dimensions of said expanded configuration substantially correspond to at least a portion of a vertebral endplate.

19. The device of claim 3 wherein the anterior and posterior dimensions of said expanded configuration reestablish a normal curvature of the spine.

20. The device of claim 1 wherein said structure is self-expanding.

21. The device of claim 2 wherein at least one element is made of a shape memory material.

22. The device of claim 21 wherein the shape memory material is Nitinol.

23. The device of claim 22 wherein the shape memory material is temperature responsive.

24. The device of claim 1 wherein said structure is bioresorbable.

25. The device of claim 2 wherein at least one element is made of a material that selected from a group of corrosive metal or corrosive metal alloys.

26. The device of claim 15 wherein said filling material includes a hardenable material.

27. The device of claim 15 wherein said filling material is a compressible liquid or gel.

28. The device of claim 15 wherein said filling material is a non-compressible liquid or gel.

29. The device of claim 15 wherein said filling material comprises at least one osteobiologic material selected from the group consisting of BMP/bone morphogenetic proteins, LMP/LIM mineralization protein, and DBM/Demineralized bone matrix, growth differentiation factors (GDF), transforming growth factors (TGF), hydroxyapatite, tri-calcium phosphate (TCP), bioactive glass, calcium phosphate, calcium sulfates, collagen, alginate.

30. The device of claim 15 wherein the filling material is solid.

31. The device of claim 15 wherein the filling material is semi-solid.

32. The device of claim 15 wherein said filling material comprises a material selected from the group consisting of hydroxyaptite spheres, plastic spheres, polymeric, ceramic and metal.

33. The device of claim 10 wherein a cross-sectional shape of the expanded configuration is selected from the group consisting of kidney-shaped, C-shaped, rectangular, square, cylindrical, capsule, U-shaped, V-shaped, X-shaped, oval-like, spherical and “O” or donut shaped.

34. The device of claim 10 wherein the expanded configuration of said device is O-shaped and wherein said device has a footprint substantially corresponding to a perimeter of two adjacent vertebral endplates.

35. The device of claim 1 wherein said structure has regions of differential rigidity or extensibility.

36. The device of claim 35 wherein the rigidity of an anterior area of the outer structure is lesser than the rigidity of a posterior area of said outer structure and whereupon filling the cavity of said device expansion of the anterior area is greater than expansion of the posterior area.

37. The device of claim 3 wherein the device is introduced into the intervertebral disk space in said collapsible configuration via a hollow catheter or cannula.

38. The device of claim 3 wherein the device is introduced into the intervertebral disk space using a minimally invasive approach.

39. The device of claim 15 wherein said structure is removed after injection of the filling material.

40. The device of claim 39 wherein said structure is removed in a collapsible configuration.

41. The device of claim 32 wherein said mesh structure is removed via a catheter or cannula.

42. The device of claim 1 wherein said device is used for intervertebral fusion.

43. The device of claim 1 wherein said device is used as a vertebral disc replacement.

44. A method for stabilizing two adjacent vertebrae in a spine comprising
a. comprising an expandable structure configured to expand in situ into an intervertebral disc space in at least one of a medial, a lateral, an anterior, a posterior, a superior and an inferior direction and wherein said structure restricts further expansion of at least one of the medial, lateral, anterior and posterior direction after reaching a maximum allowing further expansion in superior or inferior direction and wherein said structure defines a hollow cavity.
b. filling the hollow cavity with an a filler material thereby expanding the device in the intervertebral disc space in an extended configuration wherein said structure restricts radial expansion of said device.
